(19) United States
(12) Patent Application Publication
(10) Pub. No.: US 2021/0299021 A1
(43) Pub. Date: Sep. 30, 2021

(54) A COMPOSITION FOR LIGHTENING AND REDUCING HYPER PIGMENTATION OF SKIN

(71) Applicant: Karan Ratti, New Delhi (IN)

(72) Inventor: Karan Ratti, New Delhi (IN)

(21) Appl. No.: 16/496,759

(22) PCT Filed: Mar. 21, 2018
(86) PCT No.: PCT/IB2018/051888

§ 371 Date: Sep. 23, 2019

(30) Foreign Application Priority Data
Mar. 21, 2017 (IN) 201711009865

(51) Int. Cl. A61K 8/49; A61Q 19/02; A61K 8/34; A61K 8/35; A61K 8/9761

(52) U.S. Cl. CPC A61K 8/4926; A61Q 19/02; A61K 8/347; A61K 2800/43; A61K 8/9761; A61K 2800/522; A61K 8/35

(57) ABSTRACT

The invention relates to a novel composition of a cosmetic product for topical application. More particularly, the invention relates to a cosmetic composition for lightening of skin and reducing hyper pigmentation. In the main embodiment the present invention provides a novel composition of a cosmetic product for lightening of skin and reducing skin pigmentation comprising of three key ingredients: niacinamide, 4-substituted resorcinol, and hinokitiol (β-thujaplicin) and/or extract of the parts of trees in the family Cupressaceae. The present invention provides a novel cosmetic composition which is easy to formulate; and is mild on skin. The composition can also be used as an agent for preventing skin tanning in topical products.

### FIELD OF THE INVENTION

The invention relates to a novel composition for topical application. More particularly, the invention relates to a composition for lightening of skin and reducing hyper-pigmentation.

### BACKGROUND OF THE INVENTION

Hyper-pigmentation or excess pigmentation is an idiopathic disorder which results is an unusual increase in the production of melanin and/or its distribution in the skin. It occurs from the stimulation of melanocytes either hormonally, through inflammation from skin damage, or spontaneously due to genetic predisposition. Hyper-pigmentation can manifest in several forms including melasma, freckles, dark patches, spots, café au lait macules, post-inflammatory marks, and others.

Melanogenesis is the primary process involved in the synthesis of melanin of which tyrosinase is a key melanosomal enzyme involved. Several tyrosinase inhibiting agents are currently being used in the market including retinol, ascorbic acid, and hydroquinone. However, these agents are known to have certain disadvantages. For instance, all three are unstable in the presence of light or oxygen; hence, it is difficult to formulate cosmetic products with them.

Additionally, skin lightening or de-pigmenting compositions containing retinol or hydroquinone tend to cause redness and skin irritation. Trans-cutaneous penetration of ascorbic acid is poor and thus bio-availability is a challenge.

Several approaches involving combination of two or more agents have also been used to treat hyper-pigmentation.

U.S. Pat. No. 3,856,934A discloses formulation known as Kligman formulation which is a combination of hydroquinone, retinoic acid and the corticosteroid dexamethasone.

FR2785185B1 discloses a modification of the Kligman formulation comprising of an oil-in-water emulsion containing hydroquinone, retinoic acid, a corticoid, and psi-oryzanol in the oily phase.

However, such combinations are difficult to formulate and have other drawbacks such as redness or inflammation of skin, and peeling of skin which make skin tolerability difficult.

The present invention considers the existing drawbacks of prior art and provides a skin de-pigmenting and lightening composition which is mild and efficacious.

### OBJECT OF THE INVENTION

In view of the foregoing background, it is therefore the main object of the invention to provide a novel cosmetic composition for lightening of skin and reducing hyperpigmentation.

Another object of the invention is to provide a cosmetic composition which is easy to formulate. More specifically, the invention includes ingredients which can easily be combined together for a formulation.

Another object of the invention is to provide a composition comprising of Niacinamide, 4-substituted-resorcinol, and hinokitiol (β-thujaplicin) and/or extract of the parts of trees in the family Cupressaceae as key ingredients.

Yet another object of the invention is to provide a composition comprising of Niacinamide, 4-substituted-resorcinol, and hinokitiol (β-thujaplicin) and/or extract of the parts of trees in the family Cupressaceae as key ingredients, and a photo-protective agent, an anti-oxidant, an anti-inflammatory agent, a keratolytic agent and/or a promoter of skin desquamation as optional supplementary compounds.

Yet another object of the invention is to provide a cosmetic composition which is mild on the skin and still efficacious.

Yet another object of the invention is to provide a cosmetic composition which can be used as an anti-tanning agent in a sunscreen product.

Yet another object of the invention is to provide a cosmetic composition which can be used as a skin tan preventative agent in a topical product.

### SUMMARY OF THE INVENTION

The present invention relates to a novel composition of a cosmetic product for topical application. More particularly, the invention relates to a cosmetic composition for lightening of skin and reducing hyper pigmentation.

In the main embodiment the present invention provides a novel composition of a cosmetic product for lightening of skin and reducing skin pigmentation comprising of three key ingredients:

1) Niacinamide,

2) 4-substituted resorcinol, and

3) Hinokitiol (β-thujaplicin) and/or extract of the parts of trees in the family Cupressaceae and optional supplements:

1) a photo-protective agent,

2) an anti-oxidant/free radical scavenger,

3) an anti-inflammatory agent,

4) a keratolytic agent, and/or

5) a promoter of desquamation.

The Niacinamide is present in the composition in an amount of about 0.0001% to about 50.0%. Preferably present in an amount of about 0.1% to about 25.0% and most preferably in an amount of about 1% to about 5.0%.

The 4-substituted Resorcinol is present in the composition in an amount of about 0.0001% to about 20.0%. Preferably present in an amount of about 0.01% to about 2.0% and most preferably in an amount of about 0.1% to about 0.2%.

The hinokitiol (β-thujaplicin) is present in the composition in an amount of about 0.0001% to about 10%. Preferably present in an amount of about 0.01% to about 5% and most preferably in an amount of about 0.1% to about 1.0% and/or extract of the parts of trees in the family Cupressaceae present in the composition in an amount of about 0.0001% to about 50%, preferably present in an amount of about 0.001% to about 5% and most preferably in an amount of about 0.01% to about 5.0%.

The present invention provides a novel cosmetic composition which is easy to formulate; and mild on skin. The composition can also be used as an agent for preventing skin tanning in topical products.

### DESCRIPTION OF THE INVENTION

The present invention will now be described more fully hereinafter. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention relates to a cosmetic composition for lightening and reducing pigmentation (de-pigmentation) of skin. In the main embodiment of the invention the invention provides a composition for lightening and de-pigmentation of skin comprising of three key ingredients:

1) niacinamide,

2) 4-substituted resorcinol, and

3) hinokitiol (β-thujaplicin) and/or extract of the parts of trees in the family Cupressaceae

and optional supplements:

1) a photo-protective agent,

2) an anti-oxidant/free radical scavenger,

3) an anti-inflammatory agent,

4) a keratolytic agent, and/or

5) a promoter of desquamation.

Melanin is known to be the primary determinant of skin color in humans. Melanin synthesized by special dendritic cells known as melanocytes by a process of oxidation of the amino acid tyrosine followed by polymerization. Exposure to sunlight or other UV radiation can stimulate the melanocytes to produce more melanin, hence the so-called “tanning” reaction. The three key ingredients of the cosmetic composition are—niacinamide, 4-substituted resorcinol, and hinokitiol.

Niacinamide (vitamin B3, nicotinamide, 3-pyridinecarboxamide) is a naturally existing compound and is a biologically active form of niacin found in many root vegetables as well as in yeast. Niacinamide is known to disrupt the melanosome transfer process resulting in a down-regulation of the number of melanosomes transferred from melanocytes to keratinocytes.

The niacinamide is present in the composition in an amount of about 0.0001% to about 50.0%. Preferably present in an amount of about 0.1% to about 25.0% and most preferably in an amount of about 1% to about 5.0%.

4-substituted resorcinol is a resorcinol (also called resorcin), a benzenediol (m-dihydroxybenzene) with a substitution at the 4th position of its benzene ring. 4-substituted resorcinols are known to act as a tyrosinase inhibitors; as tyrosinase is a key enzyme involved in melanin production. Consequently, use of 4-substituted resorcinol efficiently reduces melanin production. Examples of some 4-substituted resorcinols include but are not limited to, 4-n-butyl resorcinol, 4-n-ethyl resorcinol, 4-n-hexyl resorcinol, 4-n-octyl resorcinol, 4-n-fluoro resorcinol and 2,4 difluoro resorcinol or combinations thereof.

The 4-substituted resorcinol is present in the composition in an amount of about 0.0001% to about 20.0%. Preferably present in an amount of about 0.01% to about 2.0% and most preferably in an amount of about 0.1% to about 0.2%.

Hinokitiol (β-thujaplicin; 2-Hydroxy-6-propan-2-ylcyclohepta-2,4,6-trien-1-one) is a tropolone derivative, is one of the thujaplicins and is a natural monoterpenoid found in the extracts of woods of trees in the family Cupressaceae. It is known to suppress tyrosinase activity, decrease tyrosinase protein levels, and consequently inhibit melanin synthesis.

The hinokitiol of the present application may be obtained through chemical synthesis or from extractions of parts of plants of the family Cupressaceae such as juniper, Juniperus Conferta, Hongyang, Lawson cypress, American cypress, Spanish mackerel, white cedar, Fukkenhiba, microphylla, and/or Acer buergerianm, Kakumihiba, arborvitae, dolabrata, Hinoki cypress, pine and/or cedar. The extraction, isolation, and purification of hinokitiol from the appropriate plant raw materials can be carried out by the methods that are known for a person skilled in art.

The hinokitiol (β-thujaplicin) is present in the composition in an amount of about 0.0001% to about 10%, preferably present in an amount of about 0.01% to about 5% and most preferably in the amount of about 0.1% to about 1.0% and/or as an extract of the parts of trees in the family Cupressaceae is present in the composition in an amount of about 0.0001% to about 50%. Preferably present in an amount of about 0.001% to about 5.0%) and most preferably in an amount of about 0.01% to about 5.0%).

Each of these key ingredients is known to work towards lightening and de-pigmenting the skin. However, the combination of these three key ingredients has significantly higher efficiency than any one of these ingredients individually. Hence, the present invention utilizes the synergistic properties of these three key ingredients of the composition. These key ingredients target different stages of skin pigmentation process, thereby, providing an enhanced effect on skin-lightening and de-pigmentation. The proposed cosmetic composition can also be used as a preventative composition against tanning in topical or sunscreen products.

In another embodiment, present invention provides a cosmetic composition which is easy to formulate as the key ingredients have no known photo or oxidative instability. The key ingredients have no known incompatibilities with each other and are thus easily combined to form an effective formulation which is a major advantage over already existing compositions. For further improvement in the efficiency of the cosmetic product, the composition is optionally supplemented with one or more photo-protective agent, anti-oxidant, anti-inflammatory agent, keratolytic agent, and/or promoter of desquamation including but not limited to hydroxy acids, enzymes, bacterial or fungal lysates or their extracts, extracts of plant matter, etc.

According to another embodiment, present invention provides organic photo-protective agents including, but are not limited to anthranilates; derivatives of dibenzoylmethane, cinnamic, salicylic, triazine, camphor, β,β-diphenylacrylate, benzotriazole, benzophenone, benzimidazole, benzalmalonate, imidazolines, bisbenzoazolyl, methylenebis(hydroxyphenylbenzotriazole), p-aminobenzoic acid (PABA), benzoxazole, screening polymers and/or screening silicones, similar to those disclosed in patent application WO 93/04665, 4,4-diarylbutadienes; dimers obtained from α-alkylstyrene, and mixtures thereof.

Preferably, the photo-protective agents of the organic types may be chosen from the following compounds (CTFA names, USAN names, INCI names or chemical names): 1,1-Dicarboxy(2,2′-dimethylpropyl)-4,4-diphenylbutadiene, 2,4-Bis[5-1(dimethylpropyl) benzoxazol-2-yl-(4-phenyl)imino]-6-(2-ethylhexyl)imino-1,3,5-triazine, 4-Methylbenzylidene Camphor, Benzophenone-3, Benzophenone-4, Bis-Ethylhexyloxyphenol Methoxyphenyl Triazine, Butyl Methoxydibenzoylmethane, Diethylamino Hydroxybenzoyl Hexyl Benzoate, Diethylhexyl Butamido Triazone, Disodium Phenyl Dibenzimidazole Tetrasulfonate, Drometrizole TriSiloxane, Ethylhexyl dimethyl PABA, Ethylhexyl Methoxycinnamate, Ethylhexyl Salicylate, Ethylhexyl Triazone, Homomenthyl Salicylate, Isoamyl p-Methoxycinnamate, Menthyl Anthranilate, Methylene Bis-Benzotriazolyl Tetramethylbutylphenol, Octocrylene, PEG-25 PABA, Phenylbenzimidazol Sulfonic Acid, Polysilicone-15, Terephthalylidene Dicamphor Sulfonic Acid, Tris Biphenyl Triazine Drometrizole Trisiloxane, also known as Mexoryl XL, and their mixtures.

Appropriate inorganic photo-protective agents may include, but are not limited to, pigments formed of metal oxides which may or may not be coated, for example, such as pigments formed of titanium oxide (crystalline in anatase and/or rutile form or amorphous form), zinc oxide, iron oxide, cerium oxide or zirconium oxide, all of which are well known as being photo-protective agents.

The photo-protective agents may be included in cosmetic compositions in any amount ranging from 0.10% to 50.00% by weight, preferably from 0.50% to 35.00% by weight and more preferably from 1.00% to 25.00% by weight in the composition. The composition would have a preferred Sun Protection Factor (SPF) value of at the least 10, such as 15, 20, 30, 50 or greater.

According to another embodiment, present invention provides anti-oxidants/free radical scavengers. Appropriate anti-oxidants may include, but are not limited to ascorbic acid and derivatives thereof such as sodium ascorbyl phosphate, magnesium ascorbyl phosphate, 3-O-ethyl ascorbic acid and ascorbyl glucoside; emblicanin; extracts of the plant emblica officinal is; Bis-Ethylhexyl Hydroxydimethoxy Benzyl malonate; thiotaine; enzymatic antioxidants such as superoxide dimutase, catalase and peroxiredoxins; uric acid; glutathione; melatonin; tocopherol and its derivatives, hydroquinone and its derivatives.

According to another embodiment, present invention provides anti-inflammatory agents. Appropriate anti-inflammatory agents may include, but are not limited to non-steroidal, such as ibuprofen and/or its salts, diclofenac and/or its salts, acetylsalicylic acid, acetaminophen, glycyrrhetinic acid; panthenol and its derivatives; botanical extracts with anti-oxidative effect such as extracts of parts of the plants Curcuma longa, Camellia sinensis; Glycyrrhiza glabra, Morus alba; epigallocatechin gallate; allantoin; the steroidal anti-inflammatories such as hydrocortisone, betamethasone valerate and/or clobetasol propionate or combinations thereof.

According to another embodiment, present invention provides compounds with keratolytic activity and/or skin desquamation enhancing activity. Appropriate keratolytic and/or skin desquamation enhancing agents may include, but are not limited to Hydroxy Acids, phenolic acids and others. Hydroxy acids are those which include acids with at the least one hydroxy group present in their molecular structure. Hydroxy acids can categorized as α-hydroxy acids, β-hydroxy acids and/or poly-hydroxy acids. the term “α-hydroxy acids” (as used herein) means those organic molecules which comprise a hydroxy group in the alpha to the carboxyl group, the term “β-hydroxy acids” as used herein means those organic molecules which comprise a hydroxyl group in the position beta to the carboxyl group, the term “poly-hydroxy” acids as used herein means those organic molecules which comprise more than one hydroxy group in their molecule. Hydroxy acids may also be derived naturally from various fruits, botanical parts, milk, and other such material or they may be synthetically derived.

Some examples of appropriate hydroxy acids and/or phenolic acids include, but are not limited to, lactic acid, glycolic acid, tartaric acid, malic acid, mandelic acid, gluconolactone, gluconic acid, lactobionic acid, kojic acid, azeilic acid, salicylic acids and/or citric acid and/or salts and/or combinations thereof.

According to another embodiment, enzymes or natural extracts with proteolytic activity may also be used according to the present invention. Proteases may be used which can be classified as serine proteases, cysteine proteases, threonine proteases, aspartic proteases, glutamic proteases, metalloproteases, asparagine peptide lyases and/or alternatively as acid proteases, neutral proteases and basic proteases. These proteases may be used exclusively, in combination or as part of naturally or synthetically derived extracts and/or lysates of whole or parts of plant, fungi, animal, bacteria, archaea and/or viruses and/or their fermentation products and byproducts. Some examples of proteolytic enzymes include but are not limited to papain, pepsin, elastase, tripsin and chymotrypsin. Other keratolytic agents and skin desquamation enhancing agents may include but are not limited to agents that modify differentiation and/or epidermal and dermal proliferation, such as Vitamin A or retinoic acid and its derivatives, retinol and its derivatives, vitamin D and derivatives thereof, oestradiol or other oestrogens, benzoyl peroxide, or hydroquinone;

According to another embodiment, the compositions of present invention may also include various other additives that are used commonly in various dermatological and cosmetic preparations, these additives may include but not be limited to fillers such as polyacrylamide (Nylon)microbeads and/or fibers, forms of silica, either incolloidal dispersion form, and/or organic microspheres; water or oil absorbing agents or optical modifiers such as polymethylsilsesquioxane, texturizers such as polymers of the silicon types in particular C30-45 Alkyl Cetearyl Dimethicone Crosspolymer; sequestering agents, such as EDTA salts; preservative agents such as parabens and/or copreservative agents, such as ethylhexyglycerine; coloring agents; fragrance agents; extracts of botanical or animal parts; antioxidants such as emblicanin, ascorbic acid, tocopherols and their derivatives; humectants such as sodium PCA, glycerin, methyl gluceth-20, propylene glycol, pentylene glycol orbutylene glycol; pH adjusting agents such as p-anisic acid, ascorbic acid, lactic acid, or others such as buffering agents and/or neutralizing agents; alcohols such as ethanol, skin penetration enhancers such as diethylene glycol monoethyl ether; and gelling and/or thickening agents, particularly acrylamide polymers, acrylic polymers, silicates, and naturally derived polymers such as xanthan gum and carboxymethylcellulose.

According to another embodiment, the present composition may be used with optional ingredients together in one dermatological or cosmetic formulation or the optional ingredients may be used separately in a separate formulation but both formulations packaged together to be presented as one combination product.

The composition of the present invention does not have the negative effects associated with state of the art compositions and skin lightening/de-pigmenting compositions. The efficacy of composition is due to the synergistic effect of three different agents that work at different stages in the skin pigmentation process namely melanosome transfer disruption, Tyrosinase inhibition & upstream melanogenic effecters and therefore more efficient than existing single agent solutions. The composition utilizes the common delivery methods currently being employed in the state of the art compositions.

The compositions of the present invention can be furnished in any dosage form typically employed for application topically and, specifically, in the following forms:

(i) Fluidic, lotion like or gel like dispersions,

(ii) emulsions with a consistency of a liquid or partial-liquid similar to that of milk, attained by dispersing a fatty phase into an aqueous phase (Oil/Water also written as O/W) or vice versa (Water/Oil also written as W/O),

(iii) emulsions or suspensions with a semi-solid, soft, pasty, buttery or solid consistency similar to that of a cream or gel,

(iv) multi phase emulsions (O/W/O or W/O/W),

(v) micro-emulsions,

(vi) nonionic and/or ionic type vesicular dispersions or

(vii) aqueous/wax phase type dispersions.

These above compositions can be prepared as per methods known to those with average skill in the art relating to dermatological or cosmetic formulation.

The emulsion type compositions of this invention contain oils that include but are not limited to hydrocarbon oils that may be nonvolatile or volatile, silicone oils that may be nonvolatile or volatile, vegetable oils that may be nonvolatile or volatile or animal derived oils that may be nonvolatile or volatile. These compositions may additionally contain fatty, non-oily ingredients such as fatty acids, fatty alcohols, fatty acid esters and esters of fatty alcohols, silicone gums, seed derived butters such as shea butter, kokum butter, mango butter, ethers, glycols, and other substances.

The emulsion type compositions of this invention may contain at least one compound that may act as an emulsifier. Appropriate emulsifiers which may be used as per the current invention include, but are not limited to, anionic, cationic, zwitterionic and nonionic emulsifiers.

The compositions of this invention may also contain at least one compound that may act as an emollient. Appropriate emollients include, but are not limited to, nonvolatile and volatile silicone oils, branched hydrocarbons, alcohol and carboxylic acid esters, fatty acids, glycerides and mixtures thereof. Examples of emollients which may be used as per the current invention include but are not limited to at the least one of isohexadecane, isopropyl palmitate, isononyl isononanoate, heptyl undecylenate, propylene glycol, C12-15 alkyl benzoate, liquid paraffin.

The present invention will now be better explained with the help of the example and experiment which follow, all of which are intended for illustrative purposes only and are not meant to unduly limit the scope of the invention in any way.

### Example 1

Example of Formulation:

| | Inventive Composition Example 1 | |
|---|---|---|
| PHASE | RAW MATERIAL (INCI NAMES) | % w/w |
| A | Emulsifier 1 | 3.00 |
| A | Emulsifier 2 | 1.50 |
| A | Organic Photoprotective Agent 1 | 4.00 |
| A | Organic Photoprotective Agent 2 | 4.00 |
| A | Antioxidant 1 | 0.10 |
| B | Water | q.s. |
| B | Humectant 1 | 5.00 |
| B | Disodium EDTA | 0.10 |
| B | Niacinamide | 4.00 |
| B | Emulsifier 1 | 0.50 |
| B | Thickner | 0.25 |
| B | Emulsifier 2 | 0.50 |
| C | Inorganic Photoprotective Agent 1 | 5.00 |
| C | Emmolient 1 | 5.00 |
| D | Ethanol | 1.00 |
| D | Propylene glycol | 4.00 |
| D | Hinokitiol | 1.00 |
| D | 4-n-substituted resorcinol | 0.10 |
| E | Antioxidant 2 | 0.20 |
| E | Anti-inflammatory Agent 1 | 0.20 |
| E | Coloring | q.s. |
| E | Fragrance | q.s. |
| F | Neutralizing Agent 1 | q.s. |
| | | 100 |

Process of preparing the above formulation:
1. The equipment used to process the above formulation was a custom fitted vessel comprising of a Wax phase vessel, Water phase vessel, main mixing tank with an anchor stirrer and arrangement for a high speed homogenizer, recirculation loop, vacuum and product evacuation valve.
2. Make Preparation A by combining Phase A ingredients together in a wax phase vessel and heat up to 80° C. until fully melted. Stir to make homogenous and set aside maintaining temperature.
3. Make preparation B by combining Phase B ingredients together in the water phase vessel and heat up to 80° C. with constant stirring until temperature is attained and the components are fully mixed and homogeneous. Keep aside while maintaining temperature.
4. Make preparation C by combining raw material of Phase C and mixing well. Keep aside.
5. Make preparation D by combining ingredients of Phase D and mixing well until a clear solution is obtained.
6. Combine Preparation A and B in main vessel maintaining temperature of 80° C. to make Preparation AB. Seal the vessel and initiate vacuum. Mix thoroughly using the anchor stirrer at 100 RPM, high speed homogenizer set at 2000 RPM and recirculation. Continue mixing for 20 minutes.
7. Begin dropping temperature. Once the temperature of Preparation AB reaches 65° C. suspend homogenization, recirculation and reduce speed of the anchor stirrer to 50 RPM.
8. Continue dropping temperature of Preparation AB until it reaches 40° C. Release the vacuum and add Preparation C slowly to make Preparation ABC.
9. Add Preparation D slowly to Preparation ABC to make Preparation ABCD
10. At a temperature of below 35° C. add successively, phase E ingredients to make Preparation ABCDE
11. Add appropriate quantity of Phase F ingredient to obtain a final formula with a pH of 7.0
12. Formula is cooled to 24° C. and transferred to its ultimate packaging

### Experiment

To study the skin lightening efficacy of the inventive composition an in vitro skin lightening assay was performed using Melanocyte containing pigmented Reconstructed Human Epidermis (RHEm). The purpose of the assay was to evaluate via histological staining technique (Warthin-Starry) the quantitative decrease of pigmentation in the RHEm and therefore the capacity of the inventive composition to lighten skin versus its constitutive compounds and against the widely used skin lightening agent Hydroquinone. The three individual compounds of the inventive composition namely, Hinokitiol, Niacinamide, 4-n-butyl resorcinol (chosen as the 4-substituted resorcinol) and Hydroquinone were administered systemically to individual RHEm in triplicates. The concentration at which the skin lightening efficacy of the compounds was studied was determined by running a separate cytotoxicity assay of the test compounds on Normal Human Epidermal Keratinocytes (NHEK) and Normal Human Epidermal Melanocyte (NHEM) monolayers. The highest concentration at which a compound gave greater than 80% cell viability (LID₈₀) on both NHEK and NHEM or in other words, the highest concentration at which the compounds are considered as non-cytotoxic to human epidermal keratinocytes and melanocytes was chosen.

The LID₈₀ concentrations of the constituent compounds of the inventive composition were taken together and administered systemically to a separate RHEm to determine the effectiveness of the inventive composition in lightening skin.

Results showed that at LID₈₀ concentrations, individually, each test compound of the inventive composition produced lightening of the RHEm as compared to control, which is in line with prior reported studies. However, the inventive composition showed a lightening effect greater than the additive lightening effect of its constitutive compounds showing a synergistic effect. Cytotoxicity studies of the test compounds showed that the inventive composition was less cytotoxic to epidermal cells than hydroquinone. When taken together, the results showed that the inventive composition is just as efficacious as, yet milder to the skin than the industry benchmark for skin lightening Hydroquinone.

1. A cosmetic composition for lightening of skin and reducing skin pigmentation comprising of:
1) Niacinamide in an amount of about 0.0001% to about 50.0%,
2) 4-substituted resorcinol in an amount of about 0.0001% to about 20.0%, and
3) Hinokitiol (β-thujaplicin) in an amount of about 0.0001% to about 10% and/or an extract of parts of trees in the family Cupressaceae in an amount of about 0.0001% to about 50%, and optionally,
4) at least one of a photo-protective agent,
5) at least one of an anti-oxidant/free radical scavenger,
6) at least one of an anti-inflammatory agent,
7) at least one of a keratolytic agent, and/or
8) at least one of a promoter of desquamation.

2. The cosmetic composition as claimed in claim 1, wherein the niacinamide is present in the composition in an amount of about 0.1% to about 25.0% by weight.

3. The cosmetic composition as claimed in claim 1, wherein the niacinamide is present in the composition in an amount of about 1% to about 5.0% by weight.

4. The cosmetic composition as claimed in claim 1,
wherein the 4-substituted resorcinols is selected from the group consisting of 4-n-butyl resorcinol, 4-n-ethyl resorcinol, 4-n-hexyl resorcinol, 4-n-octyl resorcinol, 4-n-fluoro resorcinol and 2,4 difluoro resorcinol and combinations thereof, and
wherein the 4-substituted resorcinol is present in the composition in an amount of about 0.01% to about 2.0% by weight.

5. (canceled)

6. The cosmetic composition as claimed in claim 4, wherein the 4-substituted resorcinol is present in the composition in an amount of about 0.1% to about 0.2% by weight.

7. The cosmetic composition as claimed in claim 1, wherein the hinokitiol (β-thujaplicin) is present in the composition in an amount of about 0.01% to about 5% by weight.

8. The cosmetic composition as claimed in claim 1, wherein the hinokitiol (β-thujaplicin) is present in the composition in an amount of about 0.1% to about 1.0% by weight.

9. The cosmetic composition as claimed in claim 1, wherein the extract of the parts of trees of the family Cupressaceae is present in the composition in an amount of about 0.001% to about 5%.

10. The cosmetic composition as claimed in claim 1, wherein the extract of the parts of trees of the family Cupressaceae is present in the composition in an amount of about 0.01% to about 5.0%.

11. The cosmetic composition as claimed in claim 1, wherein the photo-protective agent is an organic photo-protective agents selected from the group consisting of derivatives of dibenzoylmethane, cinnamic, salicylic, triazine, camphor, β,β-diphenylacrylate, benzotriazole, benzophenone, benzimidazole, benzalmalonate, imidazolines, bisbenzoazolyl, methylenebis(hydroxyphenylbenzotriazole), p-aminobenzoic acid (PABA), benzoxazole, screening polymers and/or screening silicones, 4,4-diarylbutadienes; dimers obtained from α-alkylstyrene, and mixtures thereof.

12. The cosmetic composition as claimed in claim 11, wherein the organic photo-protective agents is selected from the group consisting of 1,1-Dicarboxy(2,2′-dimethylpropyl)-4,4-diphenylbutadiene, 2,4-Bis[5-1(dimethylpropyl)benzoxazol-2-yl-(4-phenyl)imino]-6-(2-ethylhexyl)imino-1,3,5-triazine, 4-Methylbenzylidene Camphor, Benzophenone-3, Benzophenone-4, Bis-Ethylhexyloxyphenol Methoxyphenyl Triazine, Butyl Methoxy dibenzoylmethane, Diethylamino Hydroxybenzoyl Hexyl Benzoate, Diethylhexyl Butamido Triazone, Disodium Phenyl Dibenzimidazole Tetrasulfonate, Drometrizole TriSiloxane, Ethylhexyl dimethyl PABA, Ethylhexyl Methoxycinnamate, Ethylhexyl Salicylate, Ethylhexyl Triazone, Homomenthyl Salicylate, Isoamyl p-Methoxycinnamate, Menthyl Anthranilate, Methylene Bis-Benzotriazolyl Tetramethylbutylphenol, Octocrylene, PEG-25 PABA, Phenylbenzimidazol Sulfonic Acid, Polysilicone-15, Terephthalylidene Dicamphor Sulfonic Acid, Tris Biphenyl Triazine Drometrizole Trisiloxane, also known as Mexoryl XL, and their mixtures.

13. The cosmetic composition as claimed in claim 1, wherein the photo-protective agent is an inorganic photo-protective agents selected from the group consisting of pigments formed of metal oxides which may or may not be coated, such as pigments formed of titanium oxide, zinc oxide, iron oxide, cerium oxide and zirconium oxide.

14. The cosmetic composition as claimed in claim 1, wherein the photo-protective agents is present in the compositions in an amount of about 0.10% to about 50.00% by weight.

15. The cosmetic composition as claimed in claim 14, wherein the photo-protective agents is present in the compositions in an amount of about 0.50% to about 35.00% by weight, and preferably about 1.00% to about 25.00% by weight.

16. The cosmetic composition as claimed in claim 1,
wherein the anti-oxidants is selected from the group consisting of ascorbic acid and derivatives thereof such as sodium ascorbyl phosphate, magnesium ascorbyl phosphate, 3-O-ethyl ascorbic acid and ascorbyl glucoside; emblicanin; extracts of a plant emblica officianlis; bis-ethylhexyl hydroxydimethoxy benzylmalonate; thiotaine; enzymatic antioxidants such as superoxide dimutase, catalase and peroxiredoxins; uric acid; glutathione; melatonin; tocopherol and its derivatives, and hydroquinone and its derivatives, and
wherein the anti-inflammatory agents is selected from the group consisting of non-steroidal, such as ibuprofen and/or its salts, diclofenac and/or its salts, acetylsalicylic acid, acetaminophen, and glycyrrhetinic acid; panthenol and its derivatives; botanical extracts with anti-oxidative effect such as extracts of parts of a plants Curcuma longa, Camellia sinensis, Glycyrrhiza glabra, and Moms alba; epigallocatechin gallate; allantoin; steroidal anti-inflammatories such as hydrocortisone, betamethasone valerate and/or clobetasol propionate, and combinations thereof.

17. (canceled)

18. The cosmetic composition as claimed in claim 1,
wherein the composition further comprises enzymes or natural extracts with proteolytic activity, and
wherein the composition has a Sun Protection Factor (SPF) value of at least 10.

19. The cosmetic composition as claimed in claim 1, wherein the keratolytic agent and/or the promoter of desquamation are selected from the group consisting of hydroxy acids and phenolic acids.

20. The cosmetic composition as claimed in claim 19, wherein the hydroxy acids and phenolic acids are selected from the group consisting of lactic acid, glycolic acid, tartaric acid, malic acid, mandelic acid, gluconolactone, gluconic acid, lactobionic acid, kojic acid, azeilic acid, salicylic acids, citric acid, their salts and combinations thereof.

21. (canceled)

22. The cosmetic composition as claimed in claim 18,
wherein the enzymes or natural extracts with proteolytic activity include proteases selected from the group consisting of serine proteases, cysteine proteases, threonine proteases, aspartic proteases, glutamic proteases, metalloproteases, asparagine peptide lyases and/or alternatively as acid proteases, neutral proteases and basic proteases, either alone or in combination or as part of naturally or synthetically derived extracts and/or lysates of whole or parts of plant, fungi, animal, bacteria, archaea and/or viruses and/or their fermentation products and byproducts, and
wherein the proteases are selected from the group consisting of papain, pepsin, elastase, tripsin and chymotrypsin.

23. (canceled)

24. A method of lightening of skin and reducing skin pigmentation comprising applying to the skin, a cosmetic composition comprising of:
1) Niacinamide in an amount of about 0.0001% to about 50.0%,
2) 4-substituted resorcinol in an amount of about 0.0001% to about 20.0%, and
3) Hinokitiol (β-thujaplicin) in an amount of about 0.0001% to about 10% and/or an extract of parts of trees in the family Cupressaceae in an amount of about 0.0001% to about 50%, and optionally,
4) at least one of a photo-protective agent,
5) at least one of an anti-oxidant/free radical scavenger,
6) at least one of an anti-inflammatory agent,
7) at least one of a keratolytic agent, and/or
8) at least one of a promoter of desquamation.

25.-38. (canceled)
